(19) United States
(12) Patent Application Publication
(10) Pub. No.: US 2015/0250551 A1
(43) Pub. Date: Sep. 10, 2015

(54) Surgical Asset Tracking Trays

(71) Applicant: Jordan Michael Ries, Denver, CO (US)

(72) Inventor: Jordan Michael Ries, Denver, CO (US)

(21) Appl. No.: 14/197,189

(22) Filed: Mar. 4, 2014

(51) Int. Cl. A61B 19/00; B65D 25/10; A61B 19/02

(52) U.S. Cl. CPC A61B 19/44; A61B 19/0271; B65D 25/10; A61B 2019/442

(57) ABSTRACT

A tray for storing surgical assets comprises at least one asset holder associated with a surgical asset and an optically readable indicia associated with the surgical asset. When the surgical asset is stored in the at least one asset holder, the optically readable indicia is not readable.

### TECHNICAL FIELD

The present disclosure generally relates to containers for storing parts, and more particularly to a tray for storing surgical instruments and implants that includes optically readable indicia for tracking the surgical instruments and implants.

### BACKGROUND

Most surgical procedures require many instruments to perform. One common practice to manage the quantity of instruments is to mere the instruments in surgical trays. Each tray may contain a few instruments or many dozen instruments. Additionally, the trays may also include an array of implant that may be used in the surgery. Typical procedures, such as a total knee replacement, may require twenty trays. As such, a typical surgery may include 1,000 unique surgical assets.

Federal regulations can require each instrument to be catalogued, counted and verified for each procedure. Current procedures require a technician to read an etched or laser marked number on each instrument, and record the asset on a count sheet. This procedure may take many hours for each surgical procedure. Additionally, human error may increase the time to complete the task as well as result in improperly recorded trays. Therefore, a system and method for quickly identifying assets in surgical trays may eliminate some of the issues with the current method of identifying surgical assets in trays.

### SUMMARY OF THE INVENTION

Accordingly, an aspect of the invention provides a tray for storing surgical assets. The tray comprises at least one asset holder associated with a surgical asset and an optically readable indicia associated with the surgical asset. When the surgical asset is stored in the at least one asset holder, the optically readable indicia is not readable.

In further embodiments, the optically readable indicia is a bar code. Additionally, the surgical asset may optically block a reader from reading the optically readable indicia when the surgical instrument is stored in the at least one asset holder. Further, the optically readable indicia may be located on the at least one asset holder. Other embodiments may include a bottom surface and side alts extending from the bottom surface of the tray. The at least one asset holder includes a pair of arms extending from the bottom surface of the tray. The pair of arms may be configured to releasably hold the surgical asset in the tray. Additionally, a clasp may be attached to a first arm of the pair of arms. The clasp may be configured to extend between the first arm and a second arm of the pair of arms such that the clasp secures the surgical asset between an upper edge of the pair of arms and the bottom surface of the tray. The clasp may be pivotally connected to the first arm. A further embodiment may include the optically readable indicia on the clasp such that the optically readable indicia is not readable when the clasp is positioned to secure the surgical asset between the upper edge of the pair of arms and the bottom of the tray.

Another aspect of the invention may provide a system for identifying a missing surgical asset. A tray may be configured to store surgical assets. The tray comprises a plurality of optically readable indicia Each optically readable indicia is associated with a specific surgical asset. A reader may be configured to read the optically readable indicia when the surgical asset associated with the optically readable indicia is missing from the tray. The optically readable indicia is configured to not be readable when the surgical asset is stored in the tray. In other embodiments the reader may be configured to display a picture of the missing surgical asset. The optically readable indicia is a bar code.

Another aspect of the invention may provide a method for identifying a missing surgical asset. The method includes storing a plurality of surgical assets in at least one tray. Another step scans the at least one tray for a unique identifier. A step matches the unique identifier to the missing surgical asset.

Alternative embodiments may include displaying the missing surgical asset on a display. Additionally, the storing step may further comprise securing the surgical assets in the at least one tray. The securing step may comprise latching a clasp to secure the surgical asset in the tray. The method may further include the step of building a list of missing surgical assets.

### BRIEF DESCRIPTION OF THE DRAWINGS

The present disclosure will became more readily apparent from the specific description accompanied by the following drawings in which:

FIG. 1 is a top view of a surgical tray including surgical assets.

FIG. 2 is a top view of a surgical pan with some surgical assets removed.

FIG. 3 is a schematic view of a tray reader.

FIG. 4 is a top view of a surgical tray and tray reader with some of the surgical assets missing from the tray.

FIG. 5 is a flowchart of the steps to inventory surgical assets.

### DETAILED DESCRIPTION OF THE INVENTION

Turning now to the drawings, a tray for storing surgical assets is shown. The tray comprises at least one asset holder associated with a surgical asset and an optically readable indicia associated with the surgical asset. When the surgical asset is stored in the at least one asset holder, the optically readable indicia is not readable.

Embodiments of the tray may include an optically readable indicia configured as a bar code. Additionally, the surgical asset may optically block a reader from reading the optically readable indicia when the surgical instrument is stored in the at least one asset holder. Further, the optically readable indicia may be located on the at least one asset holder. Other embodiments may include a bottom surface and sidewalls extending from the bottom surface of the tray. The at least one asset holder includes a pair of arms extending from the bottom surface of the tray. The pair of arms may be configured to releasably hold the surgical asset in the tray. Additionally, a clasp may be attached to a first arm of the pair of arms. The clasp may be configured to extend between the first arm and a second arm of the pair of arms such that the clasp secures the surgical asset between an upper edge of the pair of arms and the bottom surface of the tray. The clasp may be pivotally connected to the first arm. A further embodiment may include the optically readable indicia on the clasp such that the optically readable indicia is not readable when the clasp is positioned to secure the surgical asset between the upper edge of the pair of arms and the bottom of the tray.

A system for identifying a missing surgical asset may be provided. A tray may be configured to store surgical assets. The tray comprises a plurality of optically readable indicia. Each optically readable indicia is associated with a specific surgical asset. A reader may be configured to read the optically readable indicia when the surgical asset associated with the optically readable indicia is missing from the tray. The optically readable indicia is configured to not be readable when the surgical asset is stored in the tray. In other embodiments the reader may be configured to display a picture of the missing surgical asset. The optically readable indicia is a bar code.

A method for identifying a missing surgical asset is further described below. The method includes storing a plurality of surgical assets in at least one tray. Another step scans the at least one tray for a unique identifier. A step matches the unique identifier to the missing surgical asset.

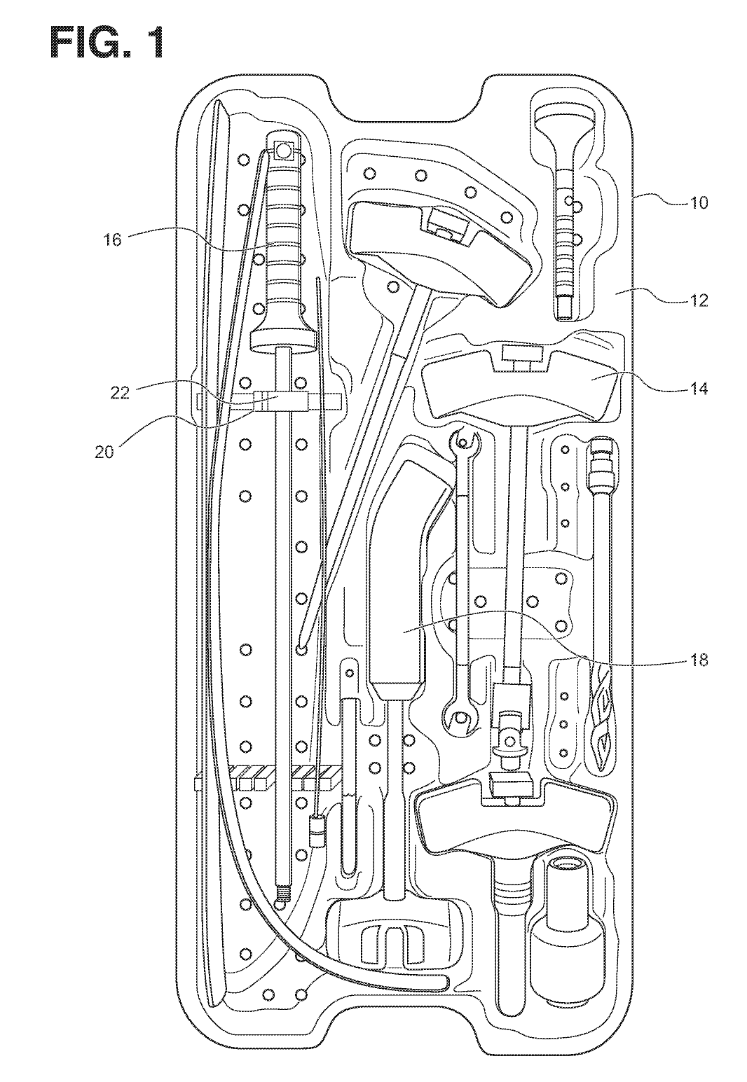

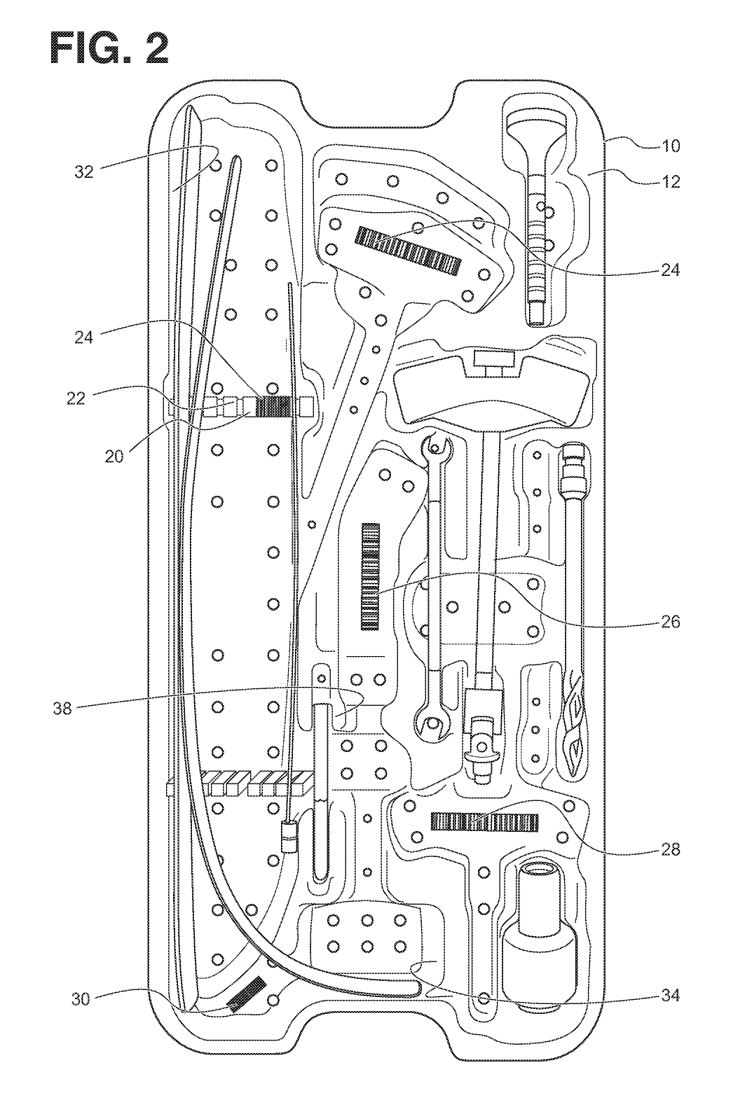

In particular, FIG. 1 shows a top view of a surgical tray 10 including surgical assets. In this embodiment, the tray 10 includes a molded pan 12. Instruments 14-18 (and many more instruments not identified by reference numbers) are retained within the tray 10. The pan 12 generally includes projections extending from a bottom surface. The projections may include the sides of the pan 12, as well as intermediate vertical projections that may create recesses in the pan (as more fully described below with reference to FIG. 2). Some instruments, which may be smaller in nature in at least one dimension may rest within arms 20 of the pan 12. Latches 22 attached to the arms 20 may close over instruments to capture the instruments in the pan 12.

The tray 10 may include multiple pans 12 laid on top of each other. The pans 12 may be stacked within the tray 10 and removed one at a time. The pan 12, while molded in this embodiment, may be constructed in any manner where specific instruments have specific orientations and positions within the pan 12. The surgical staff may then verify all instruments prior to surgery and place each instrument in its specific position in the pan 12 after surgery. The tray 10 and pan 12 may also be configured (for example, with holes) to allow for sterilization processes to run more efficiently.

Turning now to FIG. 2, FIG. 2 is a top view of a surgical pan 12 with some surgical assets removed. Optically readable indicia 24-28 are visible in the pan 2 under where the missing instruments would be located. In addition to the parts of the pan 12 shown in FIG. 1, the tray 10 in FIG. 2 also includes a registration indicia 30. Sides 32 and walls 34 of the pan 12 project from a bottom surface 36 of the pan 12. These vertical projections of the pan 12 form recesses 38 for the instruments.

The optically readable indicia 24-28 are readable when no instruments are present in the pan 12. The sides 32 and walls 34 may obstruct the view of the optically readable indicia 24-28. Placement of the indicia 24-28, then, in the recesses 38 is chosen to obstruct the view of the indicia 24-28 when the surgical instrument is properly placed in the pan 12. The indicia 24-28 will have markings or other differentiable markings to distinguish one instrument from another instrument. For example, the markings may be a one dimensional bar code, a two dimensional barcode, or other marking capable of being recognized by machine.

The arms 20 and latch 22 may also create a mechanism that allows for proper reading of an optically readable indicia 24-28. The latch 22 may be biased to remain open when no instrument is held in the arms 20 and biased close when the proper instrument is stored in the arms 20. For example, a leaf spring between the arms 22 may flip the latch 22 shut when the instrument is placed in the arms 22. The latch 22 may spring open when the instrument is pulled out. An optically readable indicia 24 may be placed under the latch 22 so that the indicia 24 is only readable when the latch is flipped open when no instrument is properly placed in the arms 20.

The sides 32 and walls 34 are positioned within the pan 12 to form the recesses 38 for the instruments. The, recesses 38 are formed to generally contour the shapes of the instruments. The indicia 24-28 in the pan 12 may then be generally covered when the instruments are properly placed in the pan 12. When an instrument is placed in the wrong position, or is otherwise missing from the pan 12, then the indicia 24-28 may be recognized by a vision system to register a missing instrument from the pan 12. Thus, the indicia 24-28, when read, register a missing instrument. By implementing such a system, the indicia 24-28 do not have to be placed on instrumentation to determine the presence of the instruments (which could also result in more easily worn indicia given the amount of handling of the instruments). Instead, the indicia 24-28 may be placed on the pan 12 which receives less stress than the instrumentation in

A registration indicia 30 may be used to identify the specific pan 12 that is being scanned. The registration indicia 20 may be useful in locating the specific indicia 24-28 within the pan 12. When the registration indicia 30 is used to locate specific indicia 24-28 within the pan 12, then a reader may be instructed to specific locations for scanning. While a single registration indicia 30 has been shown in this embodiment, multiple indicia may be used so that the tray may be presented within a reader in different orientations.

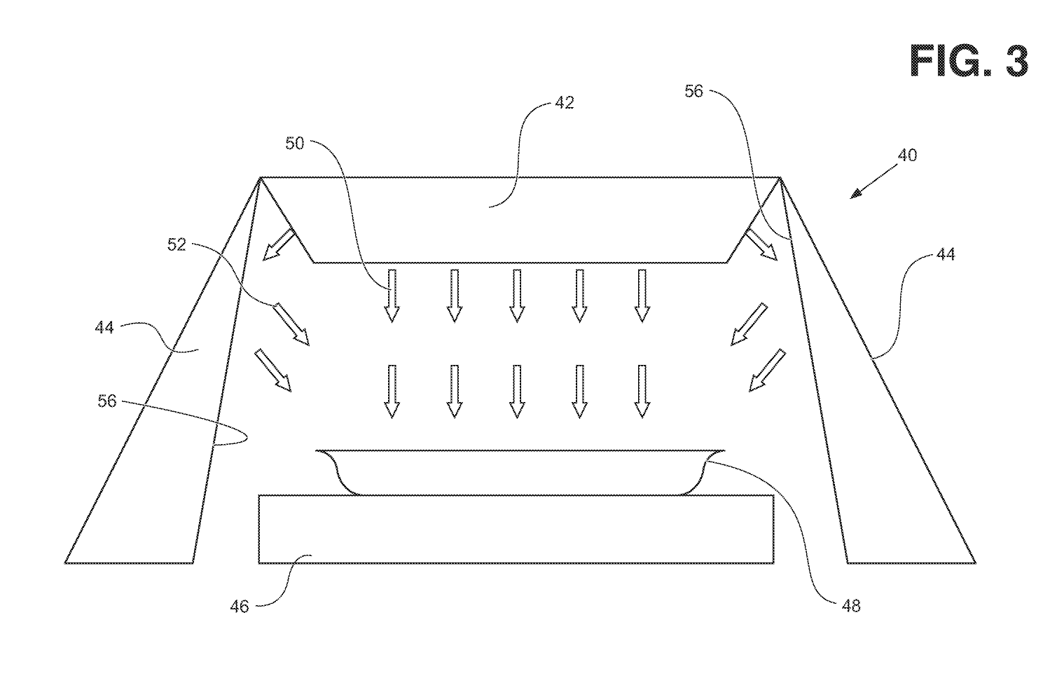

Turning now to FIG. 3, FIG. 3 is a schematic view of a tray reader 40. The reader 40 includes a reader head 42 supported by uprights 44 above a platform 46. A pan or tray 48 is placed on the platform 46 under the head 42. The head 42 may scan the tray 48 with direct sight 50 or indirect sight 52. The head 42 may be stationary with respect to the uprights 44, or may move with respect to the uprights 44 over the tray 48. The platform 46 may also move with respect to the uprights 44 or may be stationary within the frame of the reader 40.

The head 42 may be able to read the entire tray 48 with direct or indirect line of sight without moving relative to the tray 48. That may be possible based on the layout and location of the instruments within the tray 48. Deeper recesses within the tray 48 may require more indirect lines of sight to identify whether an instrument is present or missing. Shallower trays may require narrower fields of direct sight to minimize false readings returned from more angulated directions.

Indirect lines of sight 52 may be controlled through mirrored surfaces 56 on the uprights. The platform 46 and portions of the head 42 may also include mirrored portions. The positioning of the mirrored surfaces as well as the motion of the head 42 relative to the tray 48 is determined by the geometry of the tray 48 and the positioning and size of the indicia on the tray 48.

If relative motion between the tray 48 and the head 42 is necessary, the tray 48 may move relative to the reader 40. In such an instance, the platform 46 may, move relative to the tray within prescribed motions. For example, the platform 46 may linearly progress the tray 42 under the head 42. The tray 42 may also move both in and out of the plane of the reader 40 in FIG. 3 and laterally across the reader 40 in FIG. 3. Alternatively, the tray 48 may stay in place as the head 42 moves over the tray, again moving in one direction or both transverse and lateral to the tray 48. A registration indicia as shown in FIG. 2 may be used by the head 42 and platform 46 to determine which motions are necessary to properly scan the tray 10.

The reader 40 may be a device which has a single use function for reading the indicia 24-28, or may be a device that includes the reading function as one function which may be used within the device. For example, if the indicia 24-28 are bar codes, then the reader 40 may be a bar code reader. Alternatively, the reader 40 may be a smartphone or other device which may run an application which accesses the camera function of the device to recognize the indicia 24-28. In such an embodiment, the device may read the indicia 24-28 and perform other operations such as recognizing and displaying missing assets, generating reports, and communicating those reports out.

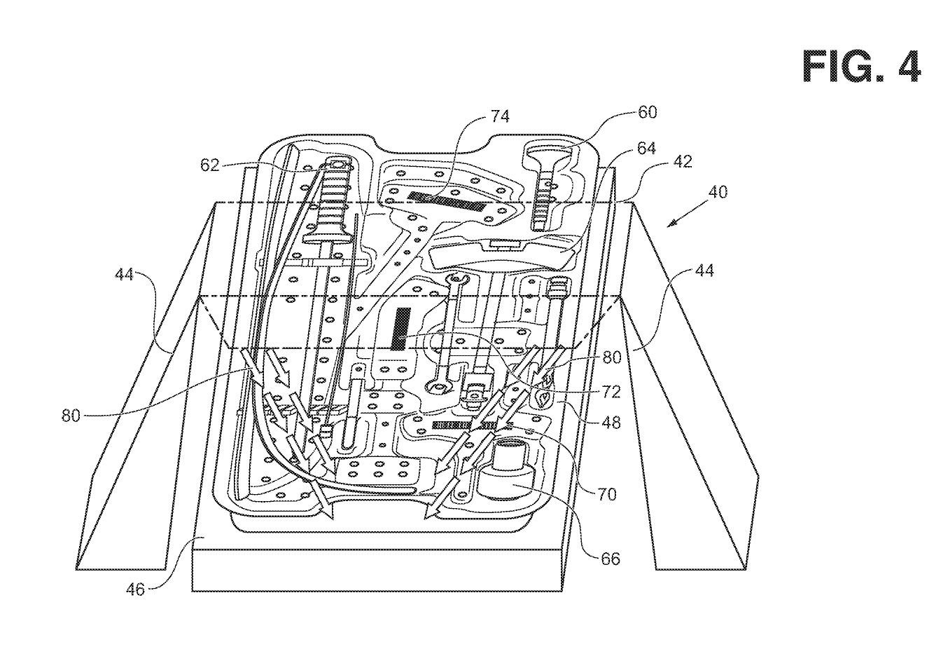

Turning now to FIG. 4, FIG. 4 is a top view of a surgical tray 48 and tray reader 40 with some of the surgical assets missing from the tray 48. Instruments 60-66 are properly stored in the tray 48. Indicia 70-74 are located on the tray 48 and are viewable because the associated instrument with the indicia is missing from the tray 48. Sight lines 80 can read the indicia 70-74. Other indicia (under the properly placed instruments) cannot be read by the reader 40 when the tray 48 is scanned. Thus, the reader, upon reading the indicia 70-74, would report the associated missing instruments to the indicia The reader 40 may report out the missing instruments as well as provide a description and picture of the missing instrument so that the technicians may search for the missing instrument. The reader 40 may also prepare a report to send to send electronically to the technician or to a reporting system to show a missing instrument.

While the system has been described with respect to instrument cases, the same may be used for surgical implants and other disposables. In those cases, the implants would not be returned to the tray, as they were part of the surgical implantation. The implants would not be missing, then, but reported as used in the surgery. The system would then report the use of the implants as part of the itemized bill for the surgery, in surgeries where the items are individually chargeable.

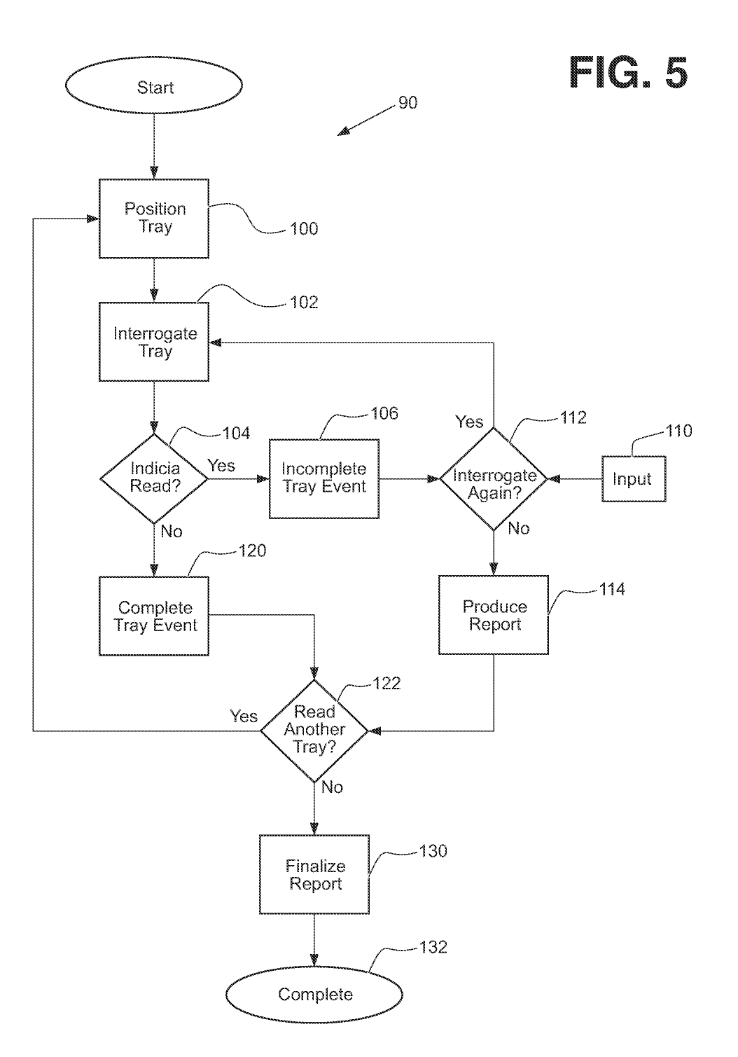

Turning now to FIG. 5, FIG. 5 is a flowchart 90 of the steps to inventory surgical assets. A tray is positioned under the reader in step 100. Step 102 interrogates the tray. A decision 104 determines whether an indicia is read. If an indicia is read an incomplete tray indicator event occurs 106. Input 110 is received to determine whether the tray should be interrogated again in step 112. If the tray should be interrogated again, then step 102 is repeated. If the tray is not interrogated, then a report is produced showing the missing instrument in step 114. If indicia is read in step 104, then a complete tray indication event 120 occurs. The system queries whether another tray should be read in step 122. If another tray should be interrogated, then the method returns to step 100. If no additional frays are to be interrogated, then a final report is prepared in step 130 and the method ends in step 132.

The tray indicator events 106 and 120 may be visual indicators, such as a green light/red light combination, or may be displayed to a screen. Additionally, the system may use audible signals to indicate the scans are completed. The indicators allow the technician to enter the input 110 into the system. For example, if an incomplete tray indicator has been shown, the technician may decide to look for the instrument prior to interrogating the tray again. Thus, when the technician received the incomplete tray indicator, the technician may stop the interrogation to replace the instruments and search for additional missing instruments prior to completing the tray interrogation. Alternatively, he may complete the tray so that the system produces a missing parts report and search for the instrument after completing all trays, so that the technician can search for all missing instruments prior to a final report. Re-interrogating the tray later with a found instrument would remove the missing instrument from the missing items report.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A tray for storing surgical assets, comprising:
at least one asset holder associated with a surgical asset; and
an optically readable indicia associated with the surgical asset;
such that when the surgical asset is stored in the at least one asset holder the optically readable indicia is not readable.

2. The tray of claim 1, wherein the optically readable indicia is a bar code.

3. The tray of claim 1, wherein the surgical asset optically blocks a reader from reading the optically readable indicia when the surgical instrument is stored in the at least one asset holder.

4. The tray of claim 3, wherein the optically readable indicia is located an the at feast one asset holder.

5. The tray of claim 1, wherein the tray includes a bottom surface and sidewalls extending from the bottom surface, the at least one asset holder includes a pair of arms extending from the bottom surface of the tray, the pair of arms configured to releasably hold the surgical asset in the tray.

6. The tray of claim 5, wherein a clasp is attached to a first arm of the pair of arms, the clasp configured to extend between the first arm and a second arm of the pair of arms such that the clasp secures the surgical asset between an upper edge of the pair of arms and the bottom surface of the tray.

7. The tray of claim 6, wherein the clasp is pivotally connected to the first arm.

8. The tray of claim 6, wherein the optically readable indicia is located on the clasp such that the optically readable indicia is not readable when the clasp is positioned to secure the surgical asset between the upper edge of the pair of arms and the bottom of the tray.

9. A system for identifying a missing surgical asset, comprising:
a tray configured to store surgical assets, the tray comprising a plurality of optically readable indicia, each optically readable indicia associated with a specific surgical asset; and
a reader configured to read the optically readable indicia when the surgical asset associated with the optically readable indicia is missing from the tray, the optically readable indicia configured to not be readable when the surgical asset is stored in the tray.

10. The system of claim 9, wherein the reader is configured to display a picture of the missing surgical asset.

11. The system of claim 9, therein the wherein the optically readable indicia is a bar code.

12. The system of claim 9, wherein the surgical asset optically blocks the reader from reading the optically readable indicia when the surgical instrument is stored in the at least one asset holder.

13. The system of claim 12, wherein the optically readable indicia is located on the at least one asset holder.

14. The system of claim 9, wherein the tray includes a bottom surface and sidewalls extending from the bottom surface, at least one asset holder includes a pair of arms extending from the bottom surface of the tray, the pair of arms configured to releasably hold the surgical asset in the tray.

15. The system of claim 14, wherein a clasp is attached to a first arm of the pair of arms, the clasp configured to extend between the first arm and a second arm of the pair of arms such that the clasp secures the surgical asset between an upper edge of the pair of arms and the bottom surface of the tray.

16. A method for identifying a missing surgical asset, comprising the steps of:
storing a plurality of surgical assets in at least one tray;
scanning the at least one tray for a unique identifier; and
matching the unique identifier to the missing surgical asset.

17. The method of claim 16, further comprising the step of displaying the missing surgical asset on a display.

18. The method of claim 16, wherein the storing step further comprises securing the surgical assets in the at least one tray.

19. The method of claim 18, wherein the securing step comprises latching a clasp to secure the surgical asset in the tray.

20. The method of claim 16, further comprising the step of building a list of missing surgical assets.
